(19) United States
(12) Patent Application Publication
(10) Pub. No.: US 2024/0341604 A1
(43) Pub. Date: Oct. 17, 2024

(54) INDIVIDUALIZED HEART FAILURE DIAGNOSTIC BASED ON COMORBIDITIES

(71) Applicant: Cardiac Pacemakers, Inc., St. Paul, MN (US)

(72) Inventors: Jonathan Bennett Shute, Eagan, MN (US); Bin Mi, Arden Hills, MN (US); Pramodsingh Hirasingh Thakur, Woodbury, MN (US)

(21) Appl. No.: 18/631,457

(22) Filed: Apr. 10, 2024

Related U.S. Application Data
(60) Provisional application No. 63/458,480, filed on Apr. 11, 2023.

(51) Int. Cl. A61B 5/0205; A61B 5/00; A61B 7/00; A61N 1/365; G16H 10/60

(52) U.S. Cl. CPC A61B 5/0205; A61B 5/7282; A61B 7/00; A61N 1/365; G16H 10/60

(57) ABSTRACT

Systems and methods for monitoring heart failure status in a patient are discussed. A medical-device system includes a storage device to store a correspondence between one or more heart failure comorbidities and corresponding one or more heart failure detection settings, and a heart failure detector circuit to detect a heart failure status of the patient. The heart failure detector circuit receives physiological information and heart failure comorbidity information of the patient, determines a detection setting for the patient based on the received comorbidity information and the stored correspondence, and detect a heart failure status using the received physiological information and the identified detection setting. A therapy circuit can deliver or adjust a heart failure therapy in response to the detected heart failure status.

### CLAIM OF PRIORITY

This application claims the benefit of U.S. Provisional Application No. 63/458,480 filed on Apr. 11, 2023, which is hereby incorporated by reference in its entirety.

### TECHNICAL FIELD

This document relates generally to medical devices, and more particularly, to systems, devices and methods for detecting and managing heart failure.

### BACKGROUND

Congestive heart failure (CHF) is a leading cause of death in the United States and globally. CHF is the loss of pumping power of the heart, and may affect left heart, right heart, or both sides of the heart, and result in the inability to deliver enough blood to meet the demands of peripheral tissues. CHF patients typically have enlarged heart with weakened cardiac muscles, resulting in reduced contractility and poor cardiac output of blood. CHF may be treated by drug therapy, or by an implantable medical device (IMD) such as for providing electrostimulation therapy. Although usually a chronic condition, CHF may occur suddenly.

Heart failure with preserved ejection fraction (HFpEF) and heart failure with reduced ejection fraction (HFrEF) are two major types of heart failures related to ejection fraction (EF). HFpEF, also known as diastolic heart failure, accounts for more than 50% of clinical heart failure cases. HFpEF occurs due to insufficient filling of the left ventricle with blood. The ventricle does not relax properly and thus is unable to fill with blood properly during the diastole, such as due to stiff and or thickened left ventricular (LV) heart muscles. However, patients with HFpEF generally have normal or near normal EF (e.g., greater than 50%). Coronary artery disease, high blood pressure, aortic stenosis, hypertrophic cardiomyopathy and pericardial disease are major causes of HFpEF. HFrEF, also known as systolic heart failure, occurs when the left ventricle fails to pump an adequate amount of oxygen-rich blood to the body, resulting a lower than normal EF (e.g., less than 40%). Heart attacks, coronary artery disease, high blood pressure, mitral regurgitation, viral myocarditis and aortic stenosis are some major cause of HFrEF.

Some IMDs are capable of monitoring CHF patients and detect events leading to worsening heart failure (WHF). These IMDs may include sensors to sense physiological signals from a patient. Frequent patient monitoring may help reduce heart failure hospitalization. Identification of patient at an elevated risk of developing WHF, such as heart failure decompensation, may help ensure timely treatment and improve prognosis and patient outcome. Identifying and safely managing the patients at elevated risk of WHF may avoid unnecessary medical interventions, hospitalization, and thereby reduce healthcare cost.

An IMD may contain electronic circuitry, such as a pulse generator, to generate and deliver electrostimulation to excitable tissues or organs, such as a heart. The electrostimulation may help restore or improve a CHF patient's cardiac performance, or rectify cardiac arrhythmias. One example of such electrostimulation therapy is resynchronization therapy (CRT) for correcting cardiac dyssynchrony in CHF patients.

### SUMMARY

Frequent monitoring of CHF patients and timely detection of intrathoracic fluid accumulation or other events indicative of heart failure decompensation status may help prevent WHF in CHF patients, hence reducing cost associated with heart failure hospitalization.

Ambulatory medical devices for monitoring heart failure patient may include implantable medical devices (IMD), subcutaneous medical devices, wearable medical devices or other external medical devices. An ambulatory medical device may be coupled to one or more physiological sensors to sense electrical activity and mechanical function of the heart. The ambulatory medical device may optionally deliver therapy, such as electrical stimulation pulses, to the patient to restore or improve patient cardiac function. Some of these devices may provide diagnostic features, such as using transthoracic impedance or other sensor signals. For example, fluid accumulation in the lungs decreases the transthoracic impedance due to the lower resistivity of the fluid than air in the lungs. The fluid accumulation may also elevate ventricular filling pressure, resulting in a louder S3 heart sound. Additionally, fluid accumulation in the lungs may irritate the pulmonary system and leads to decrease in tidal volume and increase in respiratory rate.

Identification of patient at an elevated risk of WHF may help ensure timely intervention such as device therapy or drug therapy, thereby improving the prognosis and patient outcome. On the other hand, identifying and safely managing patients with low risk of WHF may avoid unnecessary medical interventions, thereby reducing healthcare cost. Desired performance of WHF risk stratification may include one or more of a high sensitivity, a high specificity, a high positive predictive value (PPV), or a negative predictive value (NPV). The sensitivity represents an accuracy of identifying patients with relatively a high risk of WHF. The specificity represents an accuracy of identifying patients with relatively a low risk of WHF.

Echocardiography and biomarker tests (e.g., natriuretic peptide tests) are standard approaches to diagnose heart failure. The ratio of early diastolic mitral inflow velocity to early diastolic mitral annulus velocity (also known as E/e′ ratio), estimated using tissue Doppler echocardiography, has been used to evaluate the left ventricular (LV) filling pressure and LV stiffness, and to diagnose heart failure such as HFpEF. B-type natriuretic peptide (BNP) or N-terminal-pro-BNP (NT-pro-BNP)) is a protein secreted from heart muscles during hemodynamic overload, and can reflect LV end-diastolic wall stress and has been used as a marker for the evaluation of HFpEF and assessment of prognosis. Clinically, HFpEF may also be diagnosed using cardiac catheterization during exertion (e.g., exercise or other activities) to detect an exaggerated increase in LV filling pressure from baseline at rest, a hallmark signature of HFpEF.

Although these conventional echocardiography and/or biomarker tests are generally effective in diagnosing HFrEF, they can be more challenging to produce consistent and reliable outcome in diagnosing HFpEF. For example, some patients with invasively proven HFpEF nevertheless displayed normal NT-pro-BNP levels. The E/e′ ratio used as a surrogate for filling pressure, although a good indicator of large pressure differences (HF Vs normal), it may not be a reliable indicator of smaller changes in filling pressure (e.g., changes with exercise). Cardiac catheterization during exertion (to detect increase in LV filling pressure) can be difficult to deploy as a screening tool in a clinical setting. Furthermore, HFpEF can be clinically complicated with a variety of pathophysiological presentations other than diastolic dysfunction including, for example, longitudinal systolic dysfunction, chronotropic incompetence, autonomic dysfunction, endothelial dysfunction, pulmonary hypertension, abnormal atrioventricular coupling, skeletal muscle abnormalities, information, arterial stiffness, extra-cardiac causes of volume overload, among others. As such, HFpEF patients can be underdiagnosed or misdiagnosed, and do not get identified until they have had multiple episodes of acute decompensated heart failure (ADHF) with new or worsening signs and symptoms leading to hospitalization or an emergency department visit. For at least the above reasons, the present inventors have recognized that there remains a considerable need of systems and methods for diagnosing CHF, particularly HFpEF, and managing patients with such conditions.

This document discusses, among other things, a patient management system for detecting and managing heart failure in a patient, particular HFpEF. In accordance with an embodiment, a medical-device system includes a storage device and a heart failure detector circuit. The storage device can store a correspondence between one or more heart failure comorbidities and corresponding one or more heart failure detection configurations. The heart failure detector circuit can receive physiological information sensed from the patient and heart failure comorbidity information of the patient. Using the patient's comorbidity information and the stored correspondence, the heart failure detector circuit can identify for the patient a detection configuration from the stored one or more heart failure detection configurations, and detect a heart failure status in the patient using the received physiological information and the identified detection configuration. The medical- device system may include a therapy circuit to deliver or adjust a heart failure therapy in response to the detected heart failure status.

Example 1 is a medical-device system for detecting and managing heart failure in a patient, the medical-device system comprising: a storage device configured to store a correspondence between (i) one or more heart failure comorbidities and (ii) corresponding one or more heart failure detection settings; and a heart failure detector circuit configured to: receive physiological information sensed from the patient and heart failure comorbidity information of the patient; determine a detection setting for the patient based at least on the received heart failure comorbidity information and the stored correspondence; and detect a heart failure status in the patient using the received physiological information and the determined detection setting.

In Example 2, the subject matter of Example 1 optionally includes, wherein the heart failure detector circuit is configured to receive the heart failure comorbidity information from a medical record of patient referral chain.

In Example 3, the subject matter of any one or more of Examples 1-2 optionally include, wherein: the received physiological information includes heart sound information sensed from the patient; and the heart failure detector is configured to detect the heart failure status including to detect a presence or absence of a heart failure with preserved ejection fraction (HFpEF) using at least the heart sound information sensed from the patient.

In Example 4, the subject matter of any one or more of Examples 1-3 optionally include, wherein the stored one more heart failure comorbidities include at least one of: diabetes; a kidney disease; a pulmonary disease; or a cardiac disease.

In Example 5, the subject matter of any one or more of Examples 1-4 optionally include, wherein the stored one or more heart failure detection settings each include one or more sensor signals or signal metrics corresponding to the one or more heart failure comorbidities, wherein the heart failure detector is configured to detect the heart failure status in the patient using at least a portion of the received physiological information sensed from the patient that corresponds to the heart failure comorbidity information of the patient.

In Example 6, the subject matter of any one or more of Examples 1-5 optionally include, wherein to detect the heart failure status in the patient, the heart failure detector circuit is configured to: compute a composite signal index using the received physiological information and the determined heart failure detection setting; and detect the heart failure status in response to the composite signal index satisfying a specific condition.

In Example 7, the subject matter of Example 6 optionally includes, wherein the heart failure detector circuit is configured to determine or adjust a threshold value based on the received heart failure comorbidity information, and to detect the heart failure status based on a comparison between the composite signal index and the determined or adjusted threshold value.

In Example 8, the subject matter of any one or more of Examples 6-7 optionally include, wherein the heart failure detector circuit is configured to: determine or adjust weights for one or more of a plurality of signal metrics derived from the received physiological information; and compute the composite signal index using a weighted combination of the plurality of signal metrics.

In Example 9, the subject matter of any one or more of Examples 1-8 optionally include, wherein the storage device is further configured to store information about respective prevalence of the one or more heart failure comorbidities in heart failure patient population, wherein the heart failure detector circuit is configured to determine the detection setting for the patient further based on the prevalence associated with the received heart failure comorbidity information.

In Example 10, the subject matter of Example 9 optionally includes, wherein received heart failure comorbidity information includes first and second comorbidities with respective prevalence, wherein to determine the detection setting for the patient, the heart failure detector circuit is configured to select, from the stored one or more heart failure detection settings, a detection setting corresponding to one of the first or the second comorbidity having a larger prevalence.

In Example 11, the subject matter of any one or more of Examples 9- 10 optionally include, wherein the received comorbidity information includes first and second comorbidities with respective prevalence, wherein the heart failure detector circuit is configured to determine the detection setting for the patient using a combination of stored heart failure detection settings corresponding to the first and second comorbidities.

In Example 12, the subject matter of Example 11 optionally includes, wherein the stored detection settings include (i) a first detection setting employing first one or more signal metrics derived from the received physiological information and (ii) a second detection setting employing second one or more signal metrics derived from the received physiological information, wherein the heart failure detector circuit is configured to determine the detection setting for the patient using a weighted combination of the first one or more signal metrics and the second one or more signal metrics each weighted by respective prevalence.

In Example 13, the subject matter of any one or more of Examples 1-12 optionally include a processor configured to: determine, for each of the one or more heart failure comorbidities, a corresponding heart failure detection setting by modifying a base heart failure detection setting to include one or more signal metrics from a comorbidity-specific sensor; and establish the correspondence between the one or more heart failure comorbidities and the determined corresponding one or more heart failure detection settings.

In Example 14, the subject matter of Example 13 optionally includes, wherein to determine the corresponding heart failure detection setting for each of the one or more heart failure comorbidities further includes using a prevalence of the corresponding heart failure comorbidity in heart failure patient population.

In Example 15, the subject matter of any one or more of Examples 1-14 optionally include a therapy circuit configured to generate and deliver a heart failure therapy in accordance with the detected heart failure status.

Example 16 is a method for detecting and managing heart failure in a patient using a medical-device system, the method comprising: receiving physiological information and heart failure comorbidity information of the patient; receiving stored information about a correspondence between (i) one or more heart failure comorbidities and (ii) corresponding one or more heart failure detection settings; determining a detection setting for the patient based at least on the received heart failure comorbidity information and the stored correspondence; and detecting a heart failure status in the patient using the received physiological information and the determined detection setting.

In Example 17, the subject matter of Example 16 optionally includes, wherein receiving the heart failure comorbidity information is from a medical record of patient referral chain.

In Example 18, the subject matter of any one or more of Examples 16-17 optionally include, wherein the received physiological information includes heart sound information sensed from the patient, wherein detecting the heart failure status includes detecting a presence or absence of a heart failure with preserved ejection fraction (HFpEF) using at least the heart sound information sensed from the patient.

In Example 19, the subject matter of any one or more of Examples 16-18 optionally include, wherein detecting the heart failure status in the patient includes: computing a composite signal index using one or more signal metrics derived from the received physiological information and the determined heart failure detection setting; and detecting the heart failure status in response to the composite signal index satisfying a specific condition.

In Example 20, the subject matter of Example 19 optionally includes determining or adjusting a threshold value based on the received heart failure comorbidity information, wherein detecting the heart failure status is based at least on a comparison between the composite signal index and the determined or adjusted threshold value.

In Example 21, the subject matter of any one or more of Examples 19-20 optionally include, wherein computing the composition signal index includes: determining or adjusting weights for the one or more signal metrics; and computing the composite signal index using a weighted combination of the one or more signal metrics.

In Example 22, the subject matter of any one or more of Examples 16-21 optionally include, further comprising receiving information about respective prevalence of the one or more heart failure comorbidities in heart failure patient population, wherein determining the detection setting for the patient is further based on the prevalence associated with the received heart failure comorbidity information.

In Example 23, the subject matter of any one or more of Examples 16-22 optionally include: determining, for each of the one or more heart failure comorbidities, a corresponding heart failure detection setting by modifying a base heart failure detection setting to include one or more signal metrics from a comorbidity-specific sensor; and establishing the correspondence between the one or more heart failure comorbidities and the determined corresponding one or more heart failure detection settings.

Various embodiments described herein may help improve the medical technology of device-based heart failure patient management, particularly computerized diagnosis of HFpEF. The present inventors have recognized that HFpEF patients with different comorbidities may have different clinical presentations and different physiological reactions to the progression of heart failure. The comorbidity- based heart failure detection as discussed in this document involves automatic adjustment of detection algorithms or detection parameters based on the patient heart failure comorbidity, either directly or indirectly via the “point of identification” in the patient referral chain. When the patient has multiple comorbidities or develops different comorbidities, the HF detection algorithm may be automatically adjusted to adapt to different or newly developed comorbidities. By adapting HFpEF diagnosis to patient HF comorbid conditions can help recognize otherwise underdiagnosed HFpEF patients, and provide individualized management and treatment to alleviate symptoms and prevent exacerbation of comorbidities. The improved HFpEF diagnosis also may reduce hospitalization and healthcare costs associated with patient management. The systems, devices, and methods discussed in this document may also allow for more efficient device memory usage, such as by storing clinically relevant diagnostics. As fewer false positive detections of HF events are provided, device battery life may be extended; fewer unnecessary drugs and procedures may be scheduled, prescribed, or provided. Therapy titration, such as electrostimulation parameter adjustment, based on the heart failure status, may not only improve therapy efficacy and patient outcome, but may also save device power. As such, overall system cost savings may be realized.

Although the discussion in this document focuses WHF risk assessment, this is meant only by way of example and not limitation. It is within the contemplation of the inventors, and within the scope of this document, that the systems, devices, and methods discussed herein may also be used to detect, and alert occurrence of, cardiac arrhythmias, syncope, respiratory disease, or renal dysfunctions, among other medical conditions. Additionally, although systems and methods are described as being operated or exercised by clinicians, the entire discussion herein applies equally to organizations, including hospitals, clinics, and laboratories, and other individuals or interests, such as researchers, scientists, universities, and governmental agencies, seeking access to the patient data.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally an example of a heart failure monitor system configured to detect a heart failure status in a patient.

FIG. 3 illustrate generally examples of mapping patient comorbidities to corresponding heart failure detection settings.

FIG. 4 illustrates generally an example of a heart failure detection setting.

FIG. 5 illustrates generally an example of a method for detecting a heart failure status in a patient.

FIG. 6 illustrates generally a block diagram of an example machine upon which any one or more of the techniques discussed herein may perform.

### DETAILED DESCRIPTION

Disclosed herein are systems, devices, and methods for detecting and managing heart failure in a patient. A medical system includes a storage device to store a correspondence between one or more heart failure comorbidities and corresponding one or more heart failure detection settings, and a heart failure detector circuit configured to detect a heart failure status of the patient. The heart failure detector circuit can receive physiological information and heart failure comorbidity information of the patient, determine a detection setting for the patient based at least on the received comorbidity information and the stored correspondence, and detect a heart failure status (e.g. a HFpEF status) in the patient using the received physiological information and the identified detection setting. A therapy circuit to deliver or adjust a heart failure therapy in response to the detected heart failure status.

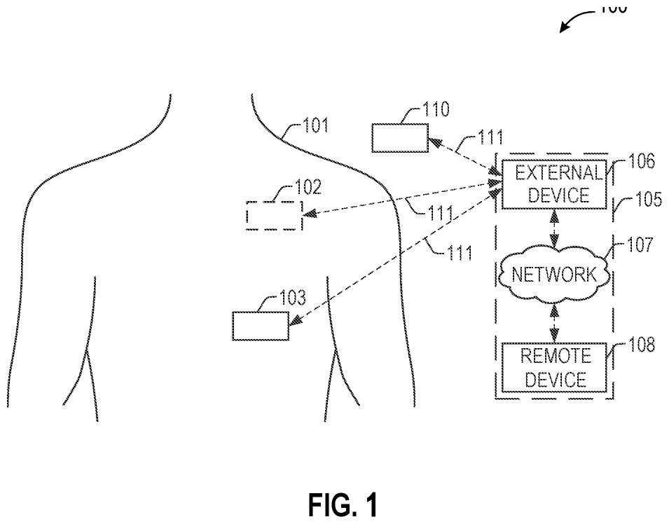

FIG. 1 illustrates generally an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 may include one or more ambulatory medical devices, an external system 105, and a communication link 111 providing for communication between the one or more ambulatory medical devices and the external system 105. The one or more ambulatory medical devices may include an implantable medical device (IMD) 102, a wearable medical device (WMD) 103, or one or more other implantable, leadless, subcutaneous, external, wearable, or ambulatory medical devices configured to monitor, sense, or detect information from, determine physiological information about, or provide one or more therapies to treat various conditions of the patient 101, such as one or more cardiac or non-cardiac conditions (e.g., dehydration, sleep disordered breathing, etc.).

In an example, the IMD 102 may include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 may include a monitor implanted, for example, subcutaneously in the chest of patient 101, the IMD 102 including a housing containing circuitry and, in certain examples, one or more sensors, such as a temperature sensor, etc.

The IMD 102 may include an assessment circuit configured to analyze specific physiological information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy may include delivery of one or more drugs to the patient 101, such as using the IMD 102 or one or more of the other ambulatory medical devices, etc. In some examples, therapy may include cardiac resynchronization therapy for rectifying dyssynchrony and improving cardiac function in heart failure patients. In other examples, the IMD 102 may include a drug delivery system, such as a drug infusion pump to deliver drugs to the patient for managing arrhythmias or complications from arrhythmias, hypertension, or one or more other physiological conditions. In other examples, the IMD 102 may include one or more electrodes configured to stimulate the nervous system of the patient or to provide stimulation to the muscles of the patient airway, etc.

The WMD 103 may include one or more wearable or external medical sensors or devices (e.g., automatic external defibrillators (AEDs), Holter monitors, patch-based devices, smart watches, smart accessories, wrist-or finger-worn medical devices, such as a finger-based photoplethysmography sensor, etc.).

In an example, the IMD 102 or the WMD 103 may include or be coupled to an implantable or wearable sensor to sense a heart sound signal, and include a heart sound recognition circuit to recognize one or more heart sound components such as S1, S2, S3, or S4. Also included in the IMD 102 or the WMD 103 is a heart sound-based event detector circuit that can detect a physiological event (e.g., a cardiac arrhythmia episode, or a heart failure event such as HFpEF event) based at least on a heart sound metric of the detected one or more heart sound component. Examples of such heart sound metric may include an amplitude, or timing of the heart sound component within a cardiac cycle relative to a fiducial point. In some examples, at least a portion of the heart sound recognition circuit and/or the heart sound-based event detector circuit may be implemented in and executed by the external system 105.

In some examples, the IMD 102 or the WMD 103 can detect and manage heart failure using physiological information sensed from the patient 101. The physiological information may be sensed using one or more ambulatory sensors included in or otherwise communicated with the IMD 102 or the WMD 103. The IMD 102 or the WMD 103 may additionally receive heart failure comorbidity information of the patient, and determine a detection setting for the patient based at least on the received comorbidity information. The detection setting for the patient may be identified from a plurality of heart failure detection settings stored in a storage device based on a predetermined correspondence between the plurality of the heart failure detection settings and the corresponding heart failure comorbidities. A detector circuit can detect a heart failure status (e.g., a HFpEF status) in the patient from the received physiological information using the identified heart failure detection setting.

The external system 105 may include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This may include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information may include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 may include an external device 106 in proximity of the one or more ambulatory medical devices, and a remote device 108 in a location relatively distant from the one or more ambulatory medical devices, in communication with the external device 106 via a communication network 107. Examples of the external device 106 may include a medical device programmer. The remote device 108 can be configured to evaluate collected patient or patient information and provide alert notifications, among other possible functions. In an example, the remote device 108 may include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server may include a memory device to store the data in a patient database. The server may include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications may include a Web page update, phone or pager call, E-mail, SMS, text or “Instant” message, as well as a message to the patient and a simultaneous direct notification to emergency services and to the clinician. Other alert notifications are possible. The server may include an alert prioritizer circuit configured to prioritize the alert notifications. For example, an alert of a detected physiological event can be prioritized using a similarity metric between the physiological data associated with the detected physiological event to physiological data associated with the historical alerts.

The remote device 108 may additionally include one or more locally configured clients or remote clients securely connected over the communication network 107 to the server. Examples of the clients may include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiological events to a system user, such as the patient or a clinician, or to a process including, for example, an instance of a computer program executable in a microprocessor. In an example, the process may include an automated generation of recommendations for anti-arrhythmic therapy, or a recommendation for further diagnostic test or treatment. In an example, the external device 106 or the remote device 108 may include a respective display unit for displaying the physiological or functional signals, or alerts, alarms, emergency calls, or other forms of warnings to signal the detection of arrhythmias. In some examples, the external system 105 may include an external data processor configured to analyze the physiological or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” may include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 may include a local external implantable medical device programmer. The external system 105 may include a remote patient management system that can monitor patient status or adjust one or more therapies such as from a remote location.

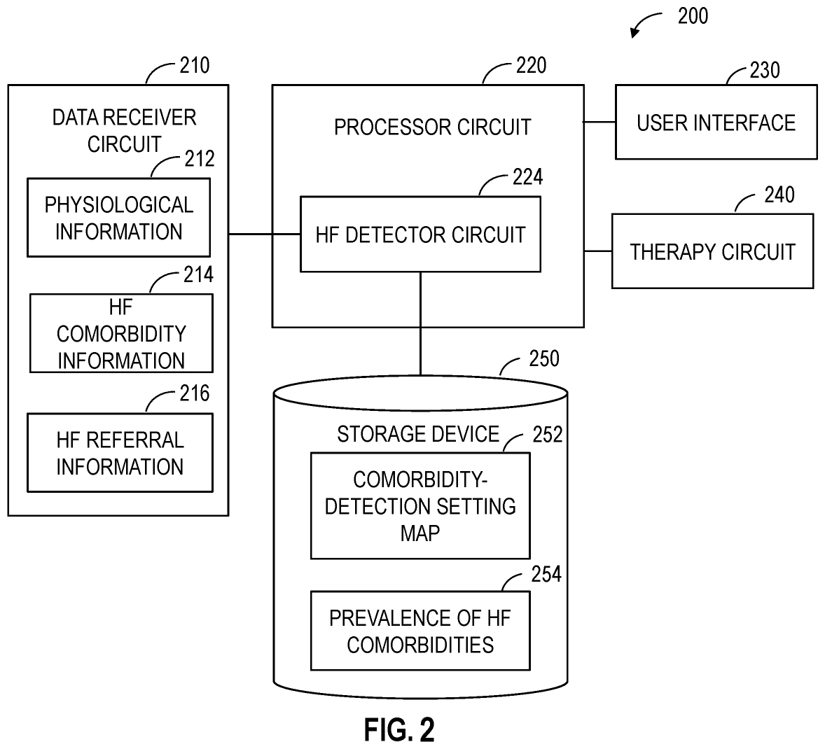

FIG. 2 illustrates generally an example of a heart failure monitor system 200 configured to detect a heart failure status in a patient. The heart failure monitor system 200 may include one or more of a data receiver circuit 210, a processor circuit 220, a user interface 230, a therapy circuit 240, and a storage device 250. At least a portion of the system 200 may be implemented in the IMD 102, the WMD 103, or the external system 105 such as one or more of the external device 106 or the remote device 108.

The data receiver circuit 210 may receive physiological information 212 from a patient. In an example, the data receiver circuit 210 may be coupled to a sensor or electrode(s) to sense the physiological information 212, such as an implantable, wearable, or otherwise ambulatory sensor or electrodes associated with the patient. The sensor may be incorporated into or associated with an ambulatory device such as the IMD 102 or the WMD 103. Examples of the physiological information 212 may include surface electrocardiography (ECG) sensed from electrodes placed on the body surface, subcutaneous ECG sensed from electrodes placed under the skin, intracardiac electrogram (EGM), heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiological response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. In some examples, the physiological information 212 sensed from a patient may be stored in a storage device, such as an electronic medical record (EMR) system. The data receiver circuit 210 may receive the physiological information 212 from the storage device, such as in response to a user command or a triggering event.

In an example, the physiological information 212 may include heart sound information sensed by a heart sound sensor from the patient. The heart sound information may include one or more of S1, S2, S3, or S4 heart sound components. In an example, the heart sound information may include a body motion/vibration signal indicative of cardiac vibration, which is correlated to or indicative of heart sounds. Examples of the heart sound sensor may include an accelerometer, an acoustic sensor, a microphone, a piezo-based sensor, or other vibrational or acoustic sensors. The accelerometer can be a one-axis, a two-axis, or a three-axis accelerometer. Examples of the accelerometer may include flexible piezoelectric crystal (e.g., quartz) accelerometer or capacitive accelerometer, fabricated using micro electro-mechanical systems (MEMS) technology. The heart sound sensor may be included in the IMD 102 or the WMD 103, or disposed on a lead such as a part of the lead system associated with the IMD 102 or the WMD 103. In an example, an accelerometer (or other sensors) may sense an epicardial or endocardial acceleration (EA) signal from a portion of a heart, such as on an endocardial or epicardial surface of one of a left ventricle, a right ventricle, a left atrium, or a right atrium. The EA signal may contain components corresponding to various heart sound components such as one or more of S1, S2, S3, or S4 components.

The data receiver circuit 210 may additionally receive one or more of heart failure comorbidity information 214 or heart failure referral information 216. Examples of the heart failure comorbidity information 214 may include diabetes, kidney dysfunction (e.g., chronic kidney disease, or CKD), pulmonary disease (e.g., chronic obstructive pulmonary disease, or COPD), cardiac arrhythmia (e.g., atrial fibrillation), cardiac diastolic dysfunctions, hypertension, autonomic dysfunctions, obesity, metabolic disorders, skeletal muscle weakness, among others.

The heart failure referral information 216 includes a “point of identification” of HFpEF patients with one or more comorbidities, such as a medical specialist in a patient referral chain who first see such patients to diagnose and manage various comorbidities and to make referral to heart failure specialists. Because HFpEF diagnosis is challenging and therapy options are primarily targeting comorbidity management, these patients generally do not follow a simple primary care to heart failure specialist referral pathway; instead, the HFpEF patients are usually first identified by a specialist aiming to manage a specific comorbid condition. Accordingly, the HFpEF patients are commonly identified through an exacerbation of their comorbid conditions. Such specialist in a patient referral chain serves as a “point of identification” of HFpEF patients with different comorbidities. For example, a cardiologist can be a point of identification of HFpEF patients with diastolic dysfunctions and symptoms, a electrophysiologist can be a point of identification of HFpEF patients with cardiac arrhythmias such as atrial fibrillation, a renal specialist can be a point of identification of HFpEF patients with renal dysfunction, an endocrinologist can be a point of identification of HFpEF patients with diabetes, a pulmonologist can be a point of identification of HFpEF patients with chronic obstructive pulmonary disease (COPD) or other respiratory disorders, etc. The heart failure referral information 216, or the “point of identification”, thus can be used to infer patient heart failure comorbidity information.

Other patient information received by the data receiver circuit 210 may include patient vital signs, medical history including prior medical, surgical, or treatment, dietary and physical activity patterns, patient demographics, clinical and lab test results, weight, etc. Such patient information, collectively referred to as clinical information in this document, may be provided by the user (e.g., the patient or a clinician) via the user interface 230, or alternatively or additionally be received automatically in response to a triggering event, such as a change in the medical history or medication of the patient.

The processor circuit 220 may detect a heart failure status in a patient using physiological information 212 and one or more of the heart failure comorbidity information 214 or the heart failure referral information 216. In an example, the processor circuit 220 can detect a HFpEF status. The processor circuit 220 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The processor circuit 220 may include circuit sets comprising one or more other circuits or sub-circuits which, either individually or in combination, perform the functions, methods or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The processor circuit 220 can include a heart failure detector circuit 224 that can determine or adjust a heart failure detection setting (DX*) for the patient based at least on the heart failure comorbidity information 214 or the heart failure referral information 216. A detection setting, as described in this document, may include one or more of physiological signals (e.g., sensor outputs) or signal metrics, values of one or more detection parameters, or detection architecture or algorithms. Selecting different settings may include selectively activating or deactivating a physiological sensor for sensing and acquiring respective physiological signal, selecting certain portions of a physiological signal (such as when the patient undergoes a particular physical activity level or during a particular time of day, or under other specified conditions), selecting different signal metrics. Different detection settings may differ in a detection threshold value to which a signal index or a composite index is compared to determine the heart failure status. Different detection settings may additionally or alternatively differ in detection architectures (e.g., decision trees, neural networks, support vector machines, logistic regression, naive- Bayes, random forests, deep neural networks, among others), or parameter values in such detection architectures such as weights assigned to respective signal metrics to compute a composite index. When the comorbidity has changed (e.g., improved or exacerbated) or when the patient develops new comorbidities, the heart failure detector circuit 224 can automatically adjust the heart failure detection setting accordingly. This advantageously ensures that the heart failure detection is comorbidity-specific and tailored to meet an individual patient's changing condition.

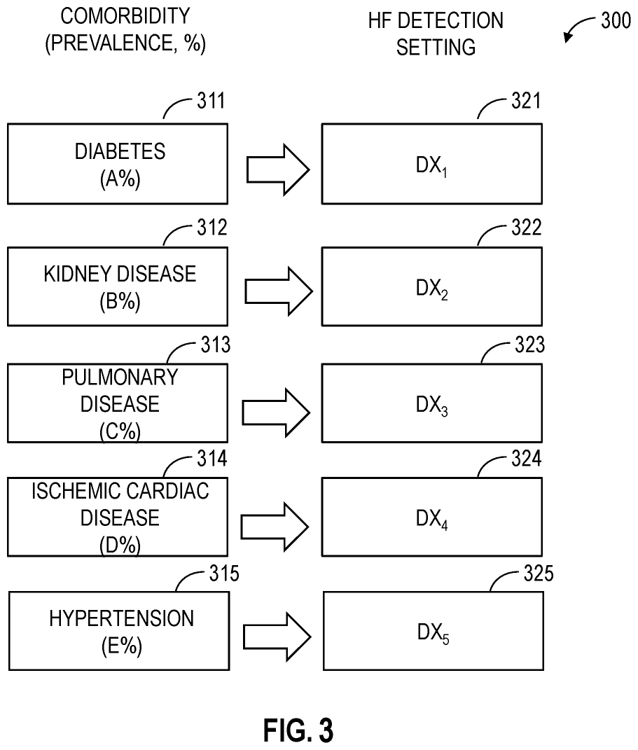

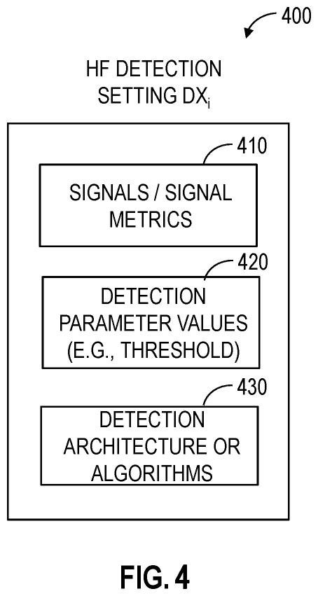

The determination or adjustment of the heart failure detection setting can be carried out using a comorbidity-detection setting map 252 stored in the storage device 250. The comorbidity-detection setting map 252 represents a correspondence between a plurality of comorbidities {C₁, C₂, . . . , C_{N}} and respective pre-determined detection settings {DX₁, DX₂, . . . , DX_{N}}. Each detection setting DXᵢ includes a detection setting optimized for detecting a heart failure status (e.g., HFpEF) in patients having the same comorbidity Cᵢ. Referring now to FIG. 3, the diagram 300 shows an example of the comorbidity-detection setting map 252, where each comorbidity is mapped to the corresponding detection setting. As similarly described above with respect to the heart failure comorbidity information 214 of the patient (as illustrated in FIG. 1), the comorbidity may include, by way of example and not limitation, one or more of diabetes 311, kidney disease 312, pulmonary disease 313, ischemic cardiac disease 314, and hypertension 315, which correspond to detection settings DX₁ 321, DX₂ 322, DX₃ 323, DX₄ 324, and DX₅ 325, respectively. FIG. 4 illustrates by way of example and not limitation a heart failure detection setting DXᵢ 400, such as any of the DX₁-DX₅ as shown in FIG. 3, which may include one or more of physiological signals (e.g., sensor outputs) or signal metrics 410, values of one or more detection parameters (such as threshold values for a signal index or a composite signal index) 420, or detection architecture or algorithms 430. The comorbidity-detection setting map may be constructed using information about comorbidities and heart failure detection performances (e.g., sensitivity, specificity, or positive predictive values) collected from a patient population having the same comorbidity. The optimal parameter setting for a particular comorbidity may be determined as one that leads to a heart failure detection performance satisfying a specific condition using data collected from the patient population having that same comorbidity.

Referring back to FIG. 2, the heart failure detector circuit 224 can identify a detection setting DX* from the commodity-detection setting map 252, and detect a heart failure status of the patient using the physiological information 212 and the detection setting DX*. In an example, the physiological information 212 include one or more physiological signals sensed using respective sensors, and the heart failure detector circuit 224 may generate one or more signal metrics from the sensed physiological signal. The signal metrics may include statistical or morphological features. By way of example and not limitation, the signal metrics may include heart rate, heart rate variability, cardiac activation timings, morphological features from the ECG or EGM, thoracic or cardiac impedance magnitude within a specified frequency range, intensities or timings of S1, S2, S3, or S4 heart sounds, systolic blood pressure, diastolic blood pressure, mean arterial pressure, or timing of a pressure metric with respect to a fiducial point, among others. In various examples, the signal metrics may be trended over time.

In an example, the heart failure detector circuit 224 may detect a heart failure status by comparing a signal metric to a detection threshold as specified in the detection setting DX*. In some examples, detection setting DX* represents a personalized, comorbidity-specific detection algorithm that computes a composite signal index (also referred to as a heart failure index) using a combination, such as a linear weighted combination, of two or more signal metrics derived from the one or more physiological signals, and determining whether the composite signal index satisfies a predetermined condition. Examples of such signal metrics may include heart sound metrics, thoracic impedance metrics, respiration metrics such as respiration rate or volume, physical activity metrics such as activity intensity or activity duration, heart rates at certain time of the day such as nocturnal heart rates, blood pressure metrics, oxygen saturation levels, cardiac arrhythmia (e.g., AF) burden, chemical or biomarker metrics, among others. A heart failure diagnosis is generated in response to the composite signal index satisfying the predetermined condition, such as exceeding a threshold. The predetermined condition, the detection threshold, and the two or more signal metrics selected for computing the composite signal index may be specified to detection setting DX*.

In the case that the detection setting includes a composite signal index weighted combination of selected signal metrics each scaled by respective weights, the heart failure detector circuit 224 can adjust the weights for respective signal metrics. Generally, signal metrics derived from sensor output that is highly correlated to a specific symptom or comorbid condition can be assigned with a higher weight. For example, for a comorbidity of COPD or other respiratory disease, the corresponding detection setting may include a larger weight assigned to respiration rate (RR) trend for constructing a composite index for heart failure detection. In another example, for a comorbidity of ischemic cardiac disease or cardiac arrhythmia (e.g., AF), the corresponding detection setting may include a larger weight assigned to AF event rate or AF burden. Yet in another example, for a comorbidity of pulmonary edema, the corresponding detection setting DX may include a larger weight assigned to total thoracic impedance for constructing a composite index for heart failure detection.

In some examples, information about prevalence of a comorbidity may be used to determine or adjust a detection setting. The prevalence herein refers to the total number of heart failure patients or a percentage of a heart failure population who have such comorbidity. The prevalence usually differs across various heart failure comorbidities, and may depend on the overlap with the particular comorbid condition. Performance of a heart failure diagnostic or a heart failure detection algorithm in a patient population can be dependent on the prevalence of one or more comorbidities. A heart failure diagnostic optimized for one comorbidity with high prevalence (e.g., hypertension with a prevalence of approximately 75% across HFpEF patients) may generally have a better overall performance in HFpEF population than a heart failure diagnostic optimized for another comorbidity with a relatively low prevalence (e.g., chronic kidney disease, or CKD, with a prevalence of approximately 20-30% in HFpEF patients).

As illustrated in FIG. 2, the storage device may store prevalence information for one or more comorbidities 254. FIG. 3 illustrates by way of example and not limitation exemplary comorbidity prevalence, such as A% (e.g., approximately 30-40%) for diabetes, B% (e.g., approximately 20-30%) for kidney disease such as CKD, C% (e.g., approximately 15-25%) for pulmonary disease such as COPD, D% (e.g., approximately 40-50%) for ischemic cardiac disease, or E% (e.g., approximately 70-80%) for hypertension, etc. The heart failure detector circuit 224 can determine the detection setting DX* for the patient further based on the prevalence of the heart failure comorbidity information 214. In an example, if the patient has multiple different comorbidities with respective different prevalence (e.g., hypertension and CKD), the heart failure detector circuit 224 may determine the detection setting DX* for the patient by selecting, from the stored heart failure detection settings, a detection setting that corresponds to the comorbidity having a larger prevalence (such as DX₅ that corresponds to hypertension which has a higher prevalence than CKD). Alternatively, the detection setting DX* may be determined to be a combination of the failure detection settings corresponding to the co-existing comorbidities (e.g., DX₂ corresponding to CKD and DX₅ corresponding to hypertension). In an example where the stored detection settings each employ respective groups of one or more signal metrics derived from the received physiological information, the heart failure detector circuit 224 may determine the detection setting DX* using a weighted combination of the respective groups of signal metrics each weighted by respective prevalence. For example, if DX₂ (corresponding to the CKD) employs first one or more signal metrics X to compute a composite signal index, and DX₅ (corresponding to the hypertension) employs second one or more signal metrics Y to compute a composite signal index, then the detection setting DX* determined for the patient may include a composite signal index(S) being calculated as a weighted combination of signal metrics X and Y, i.e., S=k₁*X+k₂*Y, where the weights k₁ and k₂ are proportional to the respective prevalence B% (for CKD) and E% (for hypertension). In an example, k₁=B%/(B%+E%), k₂=E%/(B%+E%).

In some examples, the prevalence of heart failure comorbidities 254 may be used in establishing the comorbidity-detection setting map 252. To establish the comorbidity-detection setting map 252, a base detection algorithm can be modified for different comorbidities, such as by further incorporating outputs of comorbidity- specific sensors (or signal metrics derived therefrom) that are configured to specifically detect symptoms or comorbid conditions correlated to heart failure status. By way of example and not limitation, such comorbidity-specific sensors can include AF or heart rate sensors for an AF, a blood pressure sensor or a heart sound sensor (specifically for detecting S2 heart sound morphology which is correlated to the magnitude of central aortic reflection wave predictive of HFpEF) for a hypertension, a heart sound sensor (specifically for detecting S3 heart sound) for a diastolic dysfunction, a blood chemical sensor (specifically for detecting sodium excretion as an example) for a renal dysfunction, among others. The prevalence of the comorbidities can then be used to determine, for example, weights assigned to those comorbidity-specific sensor outputs or the signal metrics derived therefrom. For a comorbidity of a higher prevalence, greater weights are assigned to signal metrics derived from the corresponding comorbidity-specific sensor output, and vice versa.

In some examples, the heart failure detector circuit 224 may process the signal metric trend and generate a predictor trend indicating temporal changes of the signal metric trend. The temporal change may be calculated using a difference between short-term values and baseline values. In an example, the short-term values may include statistical values such as a central tendency of the measurements of the signal metric within a short-term window of a first plurality of days. The baseline values may include statistical values such as a central tendency of the measurements of the signal metric within a long-term window of a second plurality of days preceding the short-term window in time. The parameters used for computing the short-term and long-term value may be specified in the comorbidity-specific detection setting DX*. In some examples, the predictor trend may be determined using a linear or nonlinear combination of the relative differences between multiple short-term values corresponding to multiple first time windows and multiple baseline values corresponding to multiple second time windows. The differences may be scaled by respective weight factors which may be based on timing information associated with corresponding multiple short-term window, such as described by Thakur et al., in U.S. Patent Publication 2017/0095160, entitled “PREDICTIONS OF WORSENING HEART FAILURE”, which is herein incorporated by reference in its entirety. In some examples, the heart failure detector circuit 224 may predict a time to heart failure event using the predictor trend. The predicted time to heart failure event may be provided to a user (e.g., a clinician) or an automated patient management system to generate a personalized, chronic patient management plan. In some examples, the heart failure detector circuit 224 may monitor patient acute responses to treatment (e.g., drug therapy or electrostimulation therapy), titrate therapy dosage, or determine patient readiness to discharge or readmission in accordance with the predictor trend.

The detected heart failure status, or a human-perceptible notification of the detection of the heart failure status, may be presented to a user via the user interface 230, such as being displayed on a display screen. Also displayed or otherwise presented to the user via the user interface 230 may include one or more of the sensed physiological signal, signal metrics, patient comorbidity information, and the comorbidity-specific detection settings DX*, among other intermediate measurements or computations. The information may be presented in a table, a chart, a diagram, or any other types of textual, tabular, or graphical presentation formats. The presentation of the output information may include audio or other media format. In an example, alerts, alarms, emergency calls, or other forms of warnings may be generated to signal the system user about the detected heart failure status.

The optional therapy circuit 240 may be configured to deliver a therapy to the patient in response to the detected heart failure status. Examples of the therapy may include electrostimulation therapy delivered to the heart, a nerve tissue, other target tissues, a cardioversion therapy, a defibrillation therapy, or drug therapy including delivering drug to a tissue or organ. In some examples, the therapy circuit 240 may modify an existing therapy, such as adjust a stimulation parameter or drug dosage.

Although the discussion herein focuses on heart failure detection, this is meant only by way of example but not limitation. Systems, devices, and methods discussed in this document may also be suitable for detecting various sorts of diseases or for assessing risk of developing other worsened conditions, such as cardiac arrhythmias, heart failure decompensation, pulmonary edema, pulmonary condition exacerbation, asthma and pneumonia, myocardial infarction, dilated cardiomyopathy, ischemic cardiomyopathy, valvular disease, renal disease, chronic obstructive pulmonary disease, peripheral vascular disease, cerebrovascular disease, hepatic disease, diabetes, anemia, or depression, among others.

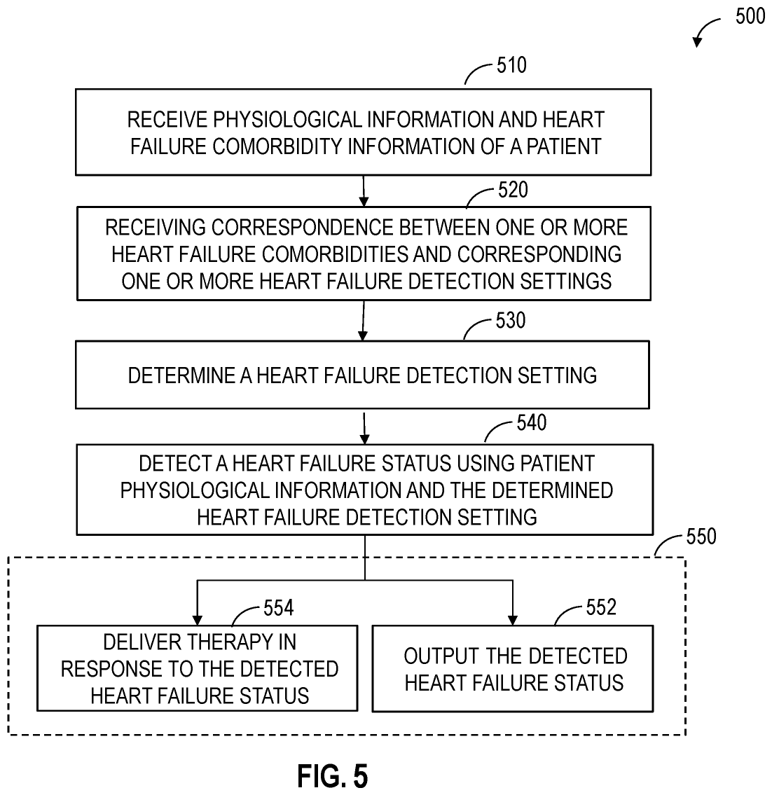

FIG. 5 illustrates generally an example of a method 500 for detecting a heart failure status in a patient. The method 500 may be implemented and executed in an ambulatory medical device, such as an implantable or wearable medical device, or in a remote patient management system. In various examples, the method 500 may be implemented in and executed by the IMD 102 or the WMD 103, one or more devices in the external system 105, or the heart failure monitor system 200 or a modification thereof.

The method 500 commences at step 510, where physiological information and heart failure comorbidity information of a patient may be received. Examples of the physiological information may include ECG, EGM, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiological response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. In an example, the physiological signal may be sensed and acquired using the sensor circuit 210 that is coupled to one or more implantable, wearable, or otherwise ambulatory sensors or electrodes associated with the patient. Alternatively, the physiological information may be acquired and stored in a storage device, such as an electronic medical record system, and may be retrieved in response to a user input or triggered by a specific event.

The received heart failure comorbidity information may include diabetes, kidney dysfunction (e.g., chronic kidney disease, or CKD), pulmonary disease (e.g., chronic obstructive pulmonary disease, or COPD), cardiac arrhythmia (e.g., atrial fibrillation), cardiac diastolic dysfunctions, hypertension, autonomic dysfunctions, obesity, metabolic disorders, skeletal muscle weakness, among others. In some examples, the heart failure comorbidity information may be inferred from heart failure referral information which may include a “point of identification” of HFpEF patients with one or more comorbidities, such as a medical specialist in a patient referral chain who first see such patients to diagnose and manage various comorbidities and to make referral to heart failure specialists.

Other patient information may also be received, including, for example, patient vital signs, medical history including prior medical, surgical, or treatment, dietary and physical activity patterns, patient demographics, clinical and lab test results, weight, etc.

At 520, a correspondence between a plurality of comorbidities {C₁, C₂, . . . , C_{N}} and respective pre-determined detection settings {DX₁, DX₂, . . . , DX_{N}} may be received. Such correspondence may be predetermined and stored in a storage device (such as storage device 250 as shown in FIG. 2) in a form of comorbidity-detection setting map. Each detection setting DXᵢ includes a detection setting optimized for detecting a heart failure status (e.g., HFpEF) in patients having the same comorbidity Cᵢ. The comorbidity-detection setting map as shown in diagram 300 may be constructed using information about comorbidities and heart failure detection performances (e.g., sensitivity, specificity, or positive predictive values) collected from a patient population having the same comorbidity. The optimal parameter setting for a particular comorbidity may be determined as one that leads to a heart failure detection performance satisfying a specific condition using data collected from the patient population having that same comorbidity. An example of the comorbidity- detection setting map and the comorbidity-specific detection setting are described above with reference to FIGS. 3-4.

At 530, a heart failure detection setting (DX*) can be determined for the patient based at least on the heart failure comorbidity information (or the heart failure referral information), and the commodity-detection setting map. A detection setting may include one or more of physiological signals (e.g., sensor outputs) or signal metrics, values of one or more detection parameters, or detection architecture or algorithms. Selecting different settings may include selectively activating or deactivating a physiological sensor for sensing and acquiring respective physiological signal, selecting certain portions of a physiological signal (such as when the patient undergoes a particular physical activity level or during a particular time of day, or under other specified conditions), selecting different signal metrics. Different detection settings may differ in a detection threshold value to which a signal index or a composite index is compared to determine the heart failure status. Different detection settings may additionally or alternatively differ in detection architectures (e.g., decision trees, neural networks, support vector machines, logistic regression, naive-Bayes, random forests, deep neural networks, among others), or parameter values in such detection architectures such as weights assigned to respective signal metrics to compute a composite index.

In some examples, information about prevalence of a comorbidity may be used to determine or adjust the detection setting. The prevalence refers to the total number of heart failure patients or a percentage of a heart failure population who have such comorbidity. The prevalence usually differs across various heart failure comorbidities, and may depend on the overlap with the particular comorbid condition. In an example, if the patient has multiple different comorbidities with respective different prevalence (e.g., hypertension and CKD), the detection setting DX* for the patient may be determined to be a detection setting that corresponds to the comorbidity having a larger prevalence. Alternatively, the detection setting DX* may be determined to be a combination of the failure detection settings corresponding to the co-existing comorbidities, such as a weighted combination of the respective groups of signal metrics each weighted by respective prevalence.

At 540, a heart failure status of the patient may be detected by applying patient physiological information to the comorbidity-specific detection setting DX*. In an example, a heart failure status may be detected by comparing a signal metric to a detection threshold as specified in the detection setting DX*. In some examples, the detection setting DX* includes computing a composite signal index (also referred to as a heart failure index) using a combination, such as a linear weighted combination, of two or more signal metrics derived from the one or more physiological signals, and determining whether the composite signal index satisfies a predetermined condition, such as exceeding a detection threshold. In some examples, detection of the heart failure status may include generating a predictor trend using a difference between short-term values and baseline values. The parameters used for computing the short-term and baseline value may be specified in the detection setting DX*. The predictor trend indicates temporal changes of the signal metric trend. Alternatively, the predictor trend may be determined using a linear or nonlinear combination of the relative differences between multiple short-term values corresponding to multiple first time windows and multiple baseline values corresponding to multiple second time windows, such as described by Thakur et al., in U.S. Patent Publication 2017/0095160, entitled “PREDICTIONS OF WORSENING HEART FAILURE”, which is herein incorporated by reference in its entirety. In an example, the heart failure status being detected may represent a particular type of heart failure, such as heart failure with preserved ejection fraction (HFpEF). HFpEF status may be detected using heart sound information sensed from the patient, such as S3 sound intensity, optionally among other physiological information. In an example, a diagnostic of HFpEF may be generated if the S3 sound intensity exceeds the comorbidity-specific S3 intensity threshold.

At 550, the detected heart failure status, or a human-perceptible notification of the detection of the heart failure status, may be presented to a user or a process. At 552, a human-perceptible presentation of the detected heart failure status may be generated and displayed to the user. Other information such as the physiological information and signal metrics derived from the physiological information, and the comorbidity information of the patient may also be displayed. The information may be presented in a table, a chart, a diagram, or any other types of textual, tabular, or graphical presentation formats. Hard copies of signals and information related to the heart failure detection may be generated. In an example, alerts, alarms, emergency calls, or other forms of warnings to signal the system user about the heart failure detection may be generated.

Additionally or alternatively, at 554, the detected heart failure status may trigger a therapy delivered to the patient, such as using the therapy circuit 240. Examples of the therapy may include electrostimulation therapy delivered to the heart, a nerve tissue, other target tissues, a cardioversion therapy, a defibrillation therapy, or drug therapy. In some examples, an existing therapy may be modified, such as by adjusting a stimulation parameter or drug dosage.

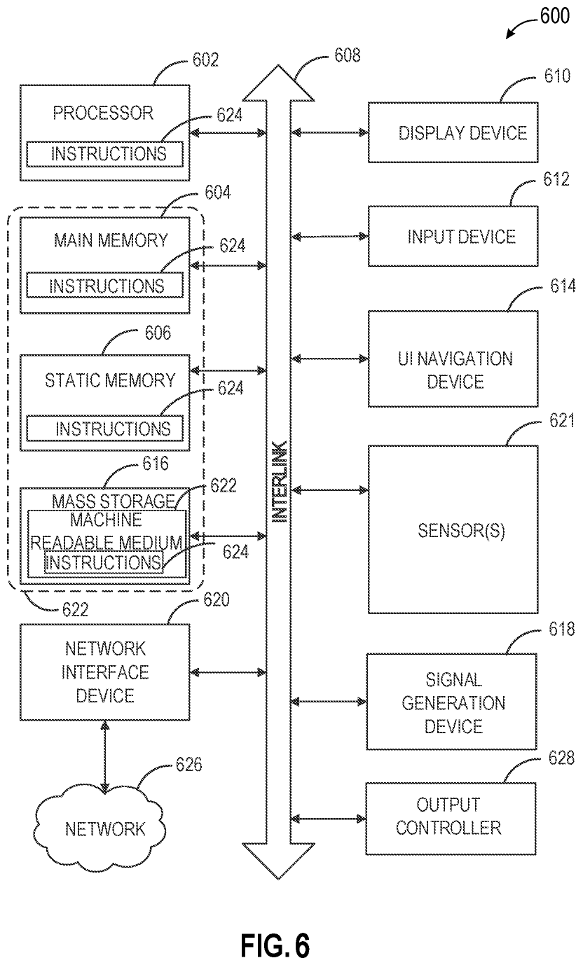

FIG. 6 illustrates generally a block diagram of an example machine 600 upon which any one or more of the techniques (e.g., methodologies) discussed herein may perform. Portions of this description may apply to the computing framework of various portions of the IMD 102, the WMD 103, the external system 105, or the heart failure monitor system 200.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specific operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine-readable media.

While the machine-readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical-device system for detecting and managing heart failure in a patient, the medical-device system comprising:
a storage device configured to store a correspondence between (i) one or more heart failure comorbidities and (ii) corresponding one or more heart failure detection settings; and
a heart failure detector circuit configured to:
receive physiological information sensed from the patient and heart failure comorbidity information of the patient;
determine a detection setting for the patient based at least on the received heart failure comorbidity information and the stored correspondence; and
detect a heart failure status in the patient using the received physiological information and the determined detection setting.

2. The medical-device system of claim 1, wherein the heart failure detector circuit is configured to receive the heart failure comorbidity information from a medical record of patient referral chain.

3. The medical-device system of claim 1, wherein:
the received physiological information includes heart sound information sensed from the patient; and
the heart failure detector is configured to detect the heart failure status including to detect a presence or absence of a heart failure with preserved ejection fraction (HFpEF) using at least the heart sound information sensed from the patient.

4. The medical-device system of claim 1, wherein the stored one or more heart failure detection settings each include one or more sensor signals or signal metrics corresponding to the one or more heart failure comorbidities,
wherein the heart failure detector is configured to detect the heart failure status in the patient using at least a portion of the received physiological information sensed from the patient that corresponds to the heart failure comorbidity information of the patient.

5. The medical-device system of claim 1, wherein to detect the heart failure status in the patient, the heart failure detector circuit is configured to:
compute a composite signal index using the received physiological information and the determined heart failure detection setting; and
detect the heart failure status in response to the composite signal index satisfying a specific condition.

6. The medical-device system of claim 5, wherein the heart failure detector circuit is configured to determine or adjust a threshold value based on the received heart failure comorbidity information, and to detect the heart failure status based on a comparison between the composite signal index and the determined or adjusted threshold value.

7. The medical-device system of claim 5, wherein the heart failure detector circuit is configured to:
determine or adjust weights for one or more of a plurality of signal metrics derived from the received physiological information; and
compute the composite signal index using a weighted combination of the plurality of signal metrics.

8. The medical-device system of claim 1, wherein the storage device is further configured to store information about respective prevalence of the one or more heart failure comorbidities in heart failure patient population,
wherein the heart failure detector circuit is configured to determine the detection setting for the patient further based on the prevalence associated with the received heart failure comorbidity information.

9. The medical-device system of claim 8, wherein the received comorbidity information includes first and second comorbidities with respective prevalence,
wherein the heart failure detector circuit is configured to determine the detection setting for the patient using a combination of stored heart failure detection settings corresponding to the first and second comorbidities.

10. The medical-device system of claim 1, comprising a processor configured to:
determine, for each of the one or more heart failure comorbidities, a corresponding heart failure detection setting by modifying a base heart failure detection setting to include one or more signal metrics from a comorbidity-specific sensor; and
establish the correspondence between the one or more heart failure comorbidities and the determined corresponding one or more heart failure detection settings.

11. The medical-device system of claim 10, wherein to determine the corresponding heart failure detection setting for each of the one or more heart failure comorbidities further includes using a prevalence of the corresponding heart failure comorbidity in heart failure patient population.

12. The medical-device system of claim 1, comprising a therapy circuit configured to generate and deliver a heart failure therapy in accordance with the detected heart failure status.

13. A method for detecting and managing heart failure in a patient using a medical-device system, the method comprising:
receiving physiological information and heart failure comorbidity information of the patient;
receiving stored information about a correspondence between (i) one or more heart failure comorbidities and (ii) corresponding one or more heart failure detection settings;
determining a detection setting for the patient based at least on the received heart failure comorbidity information and the stored correspondence; and
detecting a heart failure status in the patient using the received physiological information and the determined detection setting.

14. The method of claim 13, wherein receiving the heart failure comorbidity information is from a medical record of patient referral chain.

15. The method of claim 13, wherein the received physiological information includes heart sound information sensed from the patient,
wherein detecting the heart failure status includes detecting a presence or absence of a heart failure with preserved ejection fraction (HFpEF) using at least the heart sound information sensed from the patient.

16. The method of claim 13, wherein detecting the heart failure status in the patient includes:
computing a composite signal index using one or more signal metrics derived from the received physiological information and the determined heart failure detection setting; and
detecting the heart failure status in response to the composite signal index satisfying a specific condition.

17. The method of claim 16, comprising determining or adjusting a threshold value based on the received heart failure comorbidity information,
wherein detecting the heart failure status is based at least on a comparison between the composite signal index and the determined or adjusted threshold value.

18. The method of claim 16, wherein computing the composition signal index includes:
determining or adjusting weights for the one or more signal metrics; and
computing the composite signal index using a weighted combination of the one or more signal metrics.

19. The method of claim 13, further comprising receiving information about respective prevalence of the one or more heart failure comorbidities in heart failure patient population,
wherein determining the detection setting for the patient is further based on the prevalence associated with the received heart failure comorbidity information.

20. The method of claim 13, comprising:
determining, for each of the one or more heart failure comorbidities, a corresponding heart failure detection setting by modifying a base heart failure detection setting to include one or more signal metrics from a comorbidity-specific sensor; and
establishing the correspondence between the one or more heart failure comorbidities and the determined corresponding one or more heart failure detection settings.
